SEQUENCE LISTING (6 sequences)

SEQ ID NO: 1 - DNA, 32 bp, Artificial Sequence
  derived from Escherichia coli
gaggattttaccatggctgaaattaccgcatc

SEQ ID NO: 2 - DNA, 32 bp, Artificial Sequence
  derived from Escherichia coli
caggcggctccttggatccaattaagactgct

SEQ ID NO: 3 - DNA, 33 bp, Artificial Sequence
  derived from Escherichia coli
gtaaggaatatacatatgtctaaagaaaaattt

SEQ ID NO: 4 - DNA, 33 bp, Artificial Sequence
  derived from Escherichia coli
tcaaaactaattagagctcaattagcccagaac

SEQ ID NO: 5 - DNA, 34 bp, Artificial Sequence
  derived from Phage Q-beta
gagactgccattcatgagtaagacagcatcttcg

SEQ ID NO: 6 - DNA, 36 bp, Artificial Sequence
  derived from Phage Q-beta
atgcttagtggtggtggtaagcttacgcctcgtgta

(19) United States
(12) Patent Application Publication
(10) Pub. No.: US 2010/0151518 A1
(43) Pub. Date: Jun. 17, 2010

(54) VECTORS AND METHODS FOR ENZYME PRODUCTION

(75) Inventors: Peter Leonard Bergquist, Ellerslie (NZ); Anwar Sunna, Mona Vale (AU)

Correspondence Address: HOWSON & HOWSON LLP, 501 OFFICE CENTER DRIVE, SUITE 210, FORT WASHINGTON, PA, 19034, US

(73) Assignee: Environmental Biotechnology CRC Pty Limited, Eveleigh, NSW (AU)

(21) Appl. No.: 12/530,042

(22) PCT Filed: Mar. 6, 2008
(86) PCT No.: PCT/AU2008/000311

§ 371 Date: Feb. 25, 2010

(30) Foreign Application Priority Data
Mar. 6, 2007 (AU) 2007901150

(51) Int. Cl. C12P 21/00; C12N 9/10; C12N 15/74

(52) U.S. Cl. 435/69.1; 435/193; 435/320.1

(57) ABSTRACT

The invention provides a method for producing a replicase EF-Ts, EF-Tu and β polypeptide subunits, the method comprising expressing in a suitable host cell the EF-Ts and EF-Tu subunits, followed by expressing the β subunit, wherein the EF-Ts and EF-Tu subunits are operably linked to a first promoter and the β subunit is operably linked to a second promoter, and wherein the first and second promoters are differentially induced.

### TECHNICAL FIELD

The present invention relates to improved methods for the production of RNA replicase enzymes and to kits and vectors for use in accordance with methods of the invention. The invention also relates to uses of replicases produced in accordance with the invention.

### BACKGROUND OF THE INVENTION

Phage Qβ is a member of a family of small lytic coliphage characterized by their possession of a single stranded RNA (ssRNA) genome. Such coliphage are typically divided into two genera, the alloleviridae and the leviviridae. Qβ is an archetypal representative of the alloleviridae, as is phage SP, whilst phage MS2 and phage GA are archetypal representatives of the leviviridae. Replication of the genomes of these coliphage requires an RNA-dependent RNA polymerase (replicase). Replicase synthesis typically occurs shortly after infection of the host cell by the phage and requires both host-encoded and phage-encoded polypeptides. Specifically, these replicases are heterotetrameric enzymes, composed of three bacterial host-encoded nucleic acid binding proteins, the ribosomal protein S1 (α subunit) and elongation factors EF-Ts and Tu, and a single phage-encoded subunit, designated the β subunit in the case of Qβ replicase, being the product of the phage replicase gene.

The ability of Qβ replicase to autocatalyse RNA replication and exponentially amplify RNA template molecules is exploited in a variety of commercial and research applications including inter alia the synthesis of recombinant RNA molecules as hybridisation probes and template molecules (used for example, in RNA detection assays), for in vitro directed molecular evolution studies and antibody production.

There is a need for simple and convenient methods for producing and purifying replicases such as Qβ, in particular methods that are amenable to large scale production processes as required for commercial applications of the enzyme.

Traditionally, Qβ replicase has been produced from Escherichia coli infected with phage Qβ. Such infection-dependent production techniques can however result in RNA contamination of the purified enzyme preparation which may interfere with assays employing the enzyme. Prior art purification techniques are also typically cumbersome and labour intensive. Alternative methods for replicase production rely on the expression of cloned replicase subunits from vectors. However a significant problem, as is commonly encountered in heterologous protein expression systems, is protein aggregation and insolubility. This is presently a particular problem with production of Qβ replicase, especially expression of the β subunit.

There remains the need for improved methods for the production of ssRNA phage replicases which overcome or at least substantially ameliorate the disadvantages of prior art methods and which provide recombinant enzyme capable of being employed in a variety of commercial and research applications.

As herein described, the present invention provides a novel regulated multiple protein expression system that eliminates the need for phage culturing and enables the production of large quantities of active replicase enzyme.

### SUMMARY OF THE INVENTION

The present invention is predicated in part on the inventors' development of a series of expression vectors and methods employing these vectors that permit the production and purification of ssRNA phage replicases which, in their native form, are heteromultimeric enzymes comprised of at least two host encoded subunits, EF-Tu and EF-Ts and one phage-encoded subunit. The phage-encoded subunit is hereinafter referred to as the β polypeptide subunit, in accordance with the nomenclature for Qβ replicase.

Accordingly, the present invention provides methods for producing replicase heterotrimers comprising EF-Ts, EF-Tu and β polypeptide subunits and to constructs and expression systems for use in such methods. In accordance with the invention the EF-Ts and EF-Tu subunits are co-expressed prior to expression of the β subunit. Optionally, an EF-TsTu dimer complex is formed prior to expression of the β subunit. Optionally, the EF-Ts and EF-Tu subunits are under the transcriptional control of a first promoter and the β subunit is under the transcriptional control of a second promoter, wherein the first and second promoters are differentially induced.

In a first aspect, the present invention provides a method for producing a replicase comprising EF-Ts, EF-Tu and β polypeptide subunits, the method comprising expressing in a suitable host cell the EF-Ts and EF-Tu subunits prior to expression of the β subunit, wherein the

EF-Ts and EF-Tu subunits are operably linked to a first promoter and the β subunit is operably linked to a second promoter, and wherein the first and second promoters are differentially induced.

Optionally, a dimeric EF-TsTu complex is formed prior to expression of the β subunit.

Typically the EF-Ts and EF-Tu subunits are encoded by polynucleotides located in a first vector, and the polynucleotide encoding the β subunit is located in a second vector. The first vector may comprise at least two copies of the first promoter and the polynucleotides encoding the EF-Ts and EF-Tu subunits may be operably linked to different copies of the first promoter.

In one embodiment the first promoter is IPTG-inducible and the second promoter is arabinose-inducible or vice versa.

The replicase may be the RNA-dependent RNA polymerase of a coliphage of the alloleviridae or leviviridae. Typically the phage is Qβ, MS2, SP or GA. In a particular embodiment the replicase is the Qβ replicase.

The host cell may be any suitable host cell, for example E. coli. In one embodiment the E. coli is a lacZY deletion mutant, such as E. coli Tuner® (DE3).

Optionally the replicase may also comprise the α subunit. The a subunit may be expressed from an expression vector or may be encoded by the host cell. Alternatively, purified a subunit may be added to the heterotrimeric replicase complex following purification.

Typically the method further comprises the step of purifying the replicase produced from the host cell. Any suitable protein purification process may be used. In one embodiment, the purification comprises the steps of cell lysis, application of the extract to an anion exchange column, subsequent application of the resulting eluant to a cation exchange column and elution of purified replicase.

In a second aspect, the present invention provides a method for the production of replicase heterotrimer comprising EF-Ts, EF-Tu and β polypeptide subunits, the method comprising:
(a) providing a first expression vector comprising polynucleotides encoding the EF-Ts and EF-Tu subunits operably linked to the same or different copies of a first inducible promoter;
(b) providing a second expression vector comprising a polynucleotide encoding the β subunit operably linked to a second inducible promoter,
   wherein the first and second promoters are differentially inducible;
(c) transforming a suitable host cell with both the first and second expression vectors;
(d) culturing host cells under conditions suitable to allow expression of the EF-Ts and EF-Tu subunits from the first expression vector; and
subsequently culturing host cells under conditions suitable to allow expression of the β subunit from the second expression vector.

Optionally, a dimeric EF-TsTu complex is formed prior to expression of the β subunit.

Optionally, the method further comprises the purification of the replicase from the host cells. Thus, in one embodiment the method comprises the further steps of:
(f) lysing the host cells and collecting the supernatant containing the replicase;
(g) applying the extract supernatant to an anion exchange column;
(h) eluting one or more fractions from the column containing the replicase;
(i) applying the fractions from (h) to a cation exchange column; and
(j) eluting purified replicase from the column.

In a third aspect, the present invention provides recombinant replicase produced in accordance with a method of the invention.

In a fourth aspect, the present invention provides a dual vector expression system for use in the production of a replicase heterotrimer comprising EF-Ts, EF-Tu and β polypeptide subunits, the system comprising a first expression vector comprising polynucleotides encoding the EF-Tu and EF-Ts subunits operably linked to the same or different copies of a first inducible promoter and a second expression vector comprising a polynucleotide encoding the β subunit operably linked to a second inducible promoter, wherein the first and second promoters are differentially inducible, and wherein in use, the EF-Ts and EF-Tu subunits are expressed prior to the β subunit.

The present invention also provides host cells comprising expression vectors as described above for use in the expression system of the invention.

The invention also contemplates uses of recombinant replicases produced in accordance with aspects and embodiments of the invention in, for example, recombinant RNA synthesis, in vitro directed evolution and antibody production.

### BRIEF DESCRIPTION OF THE DRAWINGS

Embodiments of the present invention will now be described, by way of example only, with reference to the accompanying drawings:

FIG. 1. Illustration of a regulated, multiple protein expression system according to an embodiment of the present invention.

FIG. 2. Protein induction analyzed by SDS-PAGE (4-20% gel) following subunit production as described in Example 3. Total protein expression pattern of plasmids pBAD-βsubunit (encoding the β subunit of Qβ replicase) and pACYCDuet-TsTu (encoding EF-Ts and EF-Tu) in E. coli Tuner® (DE3) cells after induction with 1.0 mM IPTG at 37° C. for 30 min and with 0.2% arabinose at 30° C. for subsequent 2 h. Cells were grown in LB medium without antibiotics.

FIG. 3. SDS-PAGE (4-20% gel) analysis of Qβ replicase subunits following sonication of E. coli Tuner® (DE3) cells containing pBAD-βsubunit and pACYCDuet-TsTu plasmids. Total protein expression is shown alongside soluble and insoluble fractions.

FIG. 4. Purification of the Qβ replicase heterotrimer complex on a HiTrap Q anion exchange column (GE Healthcare). Crude extract from E. coli Tuner® was loaded into the column (Load) and the unbound proteins (Unbound) were washed from the column (Wash) with buffer B (see Example 4) containing 150mM NaCl. The Qβ replicase complex was eluted with 250 mM NaCl (Elution). Samples were analyzed by SDS-PAGE (4-20% gel).

FIG. 5. Purification of the Qβ replicase heterotrimer complex on a HiTrap S cation-exchange column (GE Healthcare). The fraction eluted from the anion-exchange column (see FIG. 3) was adjusted with 100 mM NaCl and loaded into a cation-exchange column (Load). Unbound proteins (Unbound) were washed from the column (Wash) with buffer B (see Example 4) containing 100 mM NaCl. The Qβ replicase complex was eluted with 250 mM NaCl (Elutions 1, 2 and 3). Samples were analyzed by SDS-PAGE (4-20% gel).

FIG. 6. SDS-PAGE (4-20% gel) analysis of purified Qβ replicase heterotrimer complex (B) following removal of small molecular weight contaminants using an Amicon Ultra-15 centrifugal filter (Millipore) as per Example 4. A commercial Qβ replicase heterotetramer (A) (Epicentre Biotechnologies) was included for comparison purposes. *, Protein concentration was supplied by the manufacturer.

FIG. 7. Protein induction analyzed by SDS-PAGE (4-12% gel) following subunit production as described in Example 5. Total protein expression pattern of plasmids pBAD-βsubunit (encoding the β subunit of Qβ replicase) and pACYCDuet-TsTu (encoding EF-Ts and EF-Tu) in E. coil Tuner® (DE3) cells after induction with 0.2 mM IPTG at 20° C. for 1 h and with s 0.2% arabinose at the same temperature for a subsequent 2 h. Cells were grown in LB medium without antibiotics.

FIG. 8. SDS-PAGE (4-12% gel) analysis of purified Qβ replicase heterotrimer complex following elution from a HiTrap S cation-exchange column with 250 mM NaCl (as per Example 5). The three fractions (elutions 1, 2 and 3) were concentrated and buffer-exchanged using an Amicon Ultra-15 centrifugal filter. A commercial Qβ replicase heterotetramer (Epicentre Biotechnologies) was included for comparative purposes. Samples were loaded at 1.14 and 2.28 μg/lane. *, Protein concentration was supplied by the manufacturer.

FIG. 9. Agarose-gel electrophoresis (2%) analysis of amplified Qβ replicase reaction products. Assays of purified Qβ replicase heterotrimer produced in accordance with the invention were performed in the absence (−) and presence (+) of (MDV)-poly(+) RNA template. Reaction products are shown in lanes 4 (2.5 μl enzyme),and 5 (5.0 μl enzyme). Lane 1, negative control lacking replicase and template; lane 2, template without enzyme; lane 3, template with control enzyme (commercial Qβ replicase heterotetramer).

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

The articles “a” and “an” are used herein to refer to one or to more than one (i.e. to at least one) of the grammatical object of the article. By way of example, “an element” means one element or more than one element.

By “EF-Ts” and “EF-Tu” are meant the elongation factor polypeptide subunits of the ssRNA phage replicase, which in the native enzyme are encoded by the bacterial host of the RNA coliphage. As employed in the present invention, polynucleotides encoding EF-Ts and EF-Tu are located in vectors. These polynucleotides may encode polypeptides that are identical to the native bacterial host-encoded polypeptides, or variants or derivatives thereof.

By “β subunit” is meant the polypeptide subunit of the ssRNA replicase, which in the native enzyme is the phage-encoded subunit. This term is typically used with reference to the Qβ replicase. Herein the term is applied to the normally phage-encoded subunit of a replicase of any ssRNA coliphage producing a heteromultimeric replicase enzyme. As employed in the present invention, the polynucleotide encoding the β subunit is located in a vector. These polynucleotides may encode a polypeptide that is identical to the native phage-encoded polypeptide, or a variant or derivative thereof.

By “polynucleotide” or “nucleic acid” is meant linear sequences of nucleotides, including DNA, RNA and/or known analogues of natural nucleotides, which may be double-stranded or single-stranded.

By “polypeptide,” “peptide” or “protein” is meant a polymer of amino acids joined by peptide bonds in a specific sequence.

By “derivative” is meant a polynucleotide or polypeptide that has been derived from a reference polynucleotide or polypeptide, respectively, for example by conjugation or complexing with other chemical moieties or by post-transcriptional or post-translational modification techniques as would be understood in the art.

By “variant” is meant a polynucleotide or polypeptide displaying substantial sequence identity with a reference polynucleotide or polypeptide, respectively. Variant polynucleotides also include polynucleotides that hybridise with a reference sequence under stringent conditions. These terms also encompasses polynucleotides which differ from a reference polynucleotide by the addition, deletion or substitution of at least one nucleotide. In this regard, it is well understood in the art that certain alterations inclusive of mutations, additions, deletions and substitutions can be made to a reference polynucleotide whereby the altered polynucleotide retains the biological function or activity of the reference polynucleotide. With regard to variant polynucleotides naturally occurring allelic variants are also encompassed. With regard to variant polypeptides, it is well understood in the art for example that some amino acids may be changed to other amino acids with broadly similar properties without changing the nature of the activity of the polypeptide (conservative substitutions).

In the context of this specification, the term “expression” refers to expression of a polynucleotide and/or expression of a polypeptide. Accordingly, in some contexts, reference to expression as being in relation to a polynucleotide or in relation to a polypeptide may be interchangeable. “Expression” of a polynucleotide includes transcriptional and/or post-transcriptional events. “Expression” of a polypeptide includes translational and/or post-translational events.

By “promoter” is meant a region of DNA, generally upstream (5′) of a polynucleotide coding region, which controls at least in part the initiation and level of transcription of that polynucleotide. Reference herein to a “promoter” is to be taken in its broadest context and includes the transcriptional regulatory sequences such as operator sequences, activating sequences, is enhancers and ribosome binding sites. Promoters according to the invention may contain additional specific regulatory elements, located more distal to the start site to further enhance expression in a cell, and/or to alter the timing or inducibility of expression of a structural gene to which it is operably connected. The term “promoter” includes within its scope inducible, repressible and constitutive promoters. An inducible promoter is a promoter that is positively regulated; that is the promoter is activated in the presence of an inducer molecule or system, either directly or indirectly.

As used herein the term “differentially inducible” means that expression can be independently regulated from different promoters. That is, two promoters that are “differentially inducible” means that expression from each promoter is regulated by different inducer molecules or systems thereby allowing expression from each promoter to be controlled independently of the other, in particular temporally.

By “operably linked” is meant a linkage of polynucleotide elements in a functional relationship. Thus, a promoter is operably linked to a transcribable polynucleotide coding region if it affects the transcription of the polynucleotide, the polynucleotide being located so as to be under the regulatory control of the promoter, which then controls the transcription and optionally translation of that polynucleotide. In the construction of heterologous promoter/structural gene combinations, it is generally preferred to position a promoter or variant thereof at a distance from the transcription start site of the transcribable polynucleotide, which is approximately the same as the distance between that promoter and the gene it controls in its natural setting; i.e.: the gene from which the promoter is derived. As is known in the art, some variation in this distance can be accommodated without loss of function and in some instances variations in this distance can enhance expression.

Qβ replicase is one of the best studied examples of an RNA-dependent RNA polymerase of a ssRNA coliphage. The present invention is exemplified herein with reference to the Qβ replicase, although those skilled in the art will readily appreciate that the present invention is not limited thereto. Accordingly, whilst particular reference is made to Qβ replicase in the following description and the examples and drawings which follow, this is not to be taken as being limiting on the disclosure of the invention provided herein.

A principal problem encountered in attempts to overproduce recombinant Qβ replicase complex in E. coli is the lack of solubility of the coliphage β subunit. The production of mostly insoluble β subunit greatly reduces the concentration of the final soluble Qβ replicase formed.

Disclosed herein is an improved method for the production of Qβ replicase heterotrimer using a dual vector expression system in which the EF-Ts and EF-Tu subunits are co-expressed prior to expression of the β subunit. Thus, the present invention provides a novel regulated multiple protein expression system that eliminates the need for phage culturing and enables the production of large quantities of soluble active replicase enzyme.

Accordingly an aspect of the invention provides a method for producing a replicase such as Qβ replicase comprising EF-Ts, EF-Tu and β polypeptide subunits, the method comprising expressing in a suitable host cell the EF-Ts and EF-Tu subunits prior to expression of the β subunit, wherein the EF-Ts and EF-Tu subunits are operably linked to a first promoter and the β subunit is operably linked to a second promoter, and wherein the first and second promoters are differentially induced.

Also provided is a method for, the production of a replicase heterotrimer, such as Qβ replicase heterotrimer, comprising EF-Ts, EF-Tu and β polypeptide subunits, the method comprising: providing a first expression vector comprising polynucleotides encoding the EF-Ts and EF-Tu subunits operably linked to the same or different copies of a first inducible promoter; providing a second expression vector comprising a polynucleotide encoding the β subunit operably linked to a second inducible promoter, wherein the first and second promoters are differentially inducible; transforming a suitable host cell with both the first and second expression vectors; culturing host cells under conditions suitable to allow expression of the EF-Ts and EF-Tu subunits from the first expression vector; and subsequently culturing host cells under conditions suitable to allow expression of the β subunit from the second expression vector.

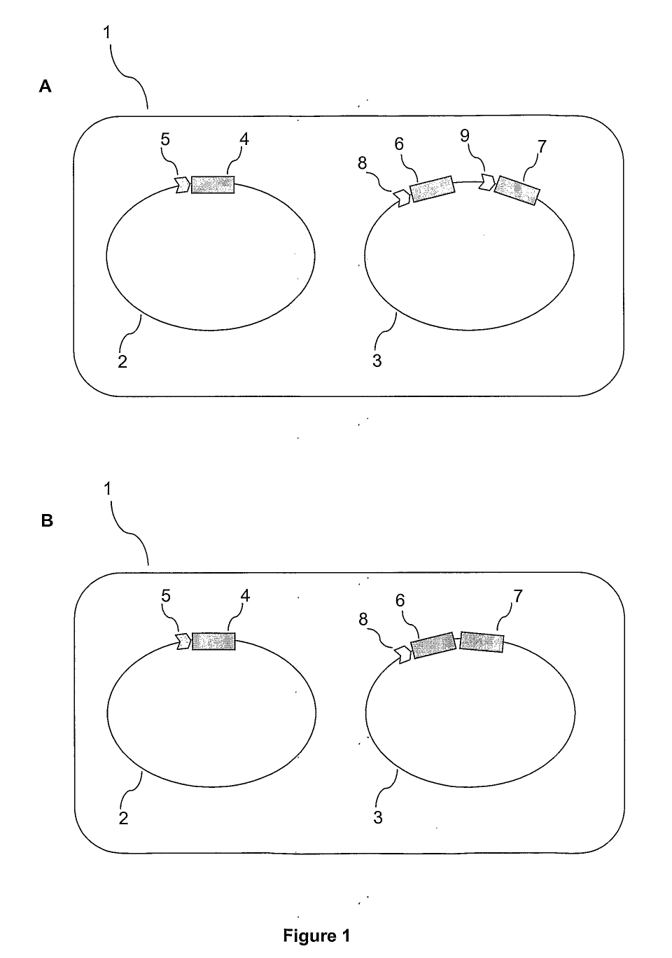

An embodiment of the present invention in accordance with the above description is illustrated in FIG. 1. It will be understood that this diagrammatic representation is provided as an example of an embodiment of the invention and in no way limits the scope of the invention.

With reference to FIG. 1, a suitable host cell 1 is transformed with expression vectors 2 and 3. The expression vectors allow for temporally regulated expression of the replicase enzyme subunits. Expression vector 2 contains a gene 4 encoding the replicase β subunit. Gene 4 is under the control of an inducible promoter 5. Expression vector 3 contains genes 6 and 7 encoding the EF-Tu and EF-Ts subunits of the replicase. In the embodiment depicted in FIG. 1A, gene 6 and gene 7 are under the control of different inducible promoters, 8 and 9. Promoters 8 and 9 may be induced by the same or different means, but are differentially induced when compared with promoter 5. In the alternative depicted in FIG. 1B, genes 6 and 7 are under the control of a single promoter 8. In the system depicted in both FIGS. 1A and 1B, in use, is expression of genes 6 and 7 occurs before expression of gene 4.

Methods of the invention enable the production of large quantities of recombinant heterotrimeric replicase without requiring phage infection of host cells, and thus avoid the problem of RNA contamination and the need for cumbersome, time consuming and labour intensive purification procedures. For example, using methods of the invention the inventors have successfully produced an approximately 10-fold greater quantity of active replicase per ml of culture medium than using prior art methods.

Methods of the invention also provide for the expression of native subunit sequences and the formation of the native polypeptides into a heterotrimeric complex in the absence of any additional or extraneous sequence modifications such as linker sequences between subunits or sequence tags to aid purification. The methods described herein do not rely on affinity tags for purification, thus reducing the possibility of aggregation problems due to tag interactions and the need for post-purification affinity tag removal.

Purification of recombinant replicase produced in accordance with the invention may be achieved using a range of protein purification techniques well known to those skilled in the art and it will be appreciated that the invention is not limited by reference to any particular means of purification. By way of non-limiting example, as exemplified herein, recombinant replicase produced in accordance with the invention may be purified by use of anion exchange and cation exchange column chromatography following cell lysis. Accordingly, the invention provides a simple and efficient method for the generation of highly pure, functional, recombinant replicase suitable for any commercial or research application for which the replicase is considered appropriate. Such applications are well known to those skilled in the art.

In many applications the heterotrimeric replicase is sufficient. However in some applications it may be necessary or desirable to incorporate the a subunit of the replicase, the ribosomal protein S1, so as to form the complete heterotetrameric enzyme. This may be achieved by any one of a number of means, either prior to or following purification of the heterotrimer. For example, purified a subunit may be added to the purified heterotrimeric complex using methods known to those skilled in the art, for example as described by Kamen et al. (1972), Reconstitution of Qβ replicase subunit alpha with protein-synthesis interference factor I. Eur. J. Biochem. 31:44-51. Alternatively, the a subunit may be expressed in the host cells containing the vectors comprising the EF-Ts, EF-Tu and β subunits such that formation of the heterotetramer occurs prior to purification. The a subunit may be encoded by the genome of the host cell or otherwise encoded by a vector.

Without wishing to be limited by any one theory or mechanism of action, it is envisaged that in accordance with the methods of the invention, following expression of the EF-Ts and EF-Tu subunits a dimeric complex between the EF-Ts and EF-Tu subunits is formed prior to expression of the β subunit and that solubility of the β subunit may be dependent upon the presence of this preformed dimeric complex. However in its broadest aspect, the invention provides for the expression of the EF-Ts and EF-Tu subunits prior to expression of the β subunit. As exemplified herein, this is typically achieved using a dual vector expression system in which polynucleotides encoding the EF-Tu and EF-Ts subunits are located on a first expression vector and operably linked to the same or different copies of a first inducible promoter, and a polynucleotide encoding the β subunit is located on a second expression vector and operably linked to a second inducible promoter, wherein the first and second promoters are differentially inducible.

Those skilled in the art will appreciate that reference to the “first' and “second” expression vectors means vectors that are compatible within the same host cell (i.e. have different origins of replication). Similarly, by “first” and “second” promoters that are differentially inducible means promoters that are able to be regulated by different mechanisms within the cell, allowing tight regulation of expression of polypeptides and in which expression from the first promoter can be initiated, and optionally switched off, prior to initiation of expression from the second promoter. As disclosed, in embodiments of the invention the first expression vector may contain at least two copies of the first promoter and the EF-Ts- and EF-Tu-encoding polynucleotides may be each located adjacent different copies of the promoter thereby eliminating the problem of differential levels of expression of the two subunits which can arise where a single promoter drives expression of multiple polypeptides.

In particular embodiments of the invention it is envisaged that the polynucleotides encoding the EF-Ts and EF-Tu subunits are typically located on the same vector. This need not be the case however, provided it is possible to coordinate expression of the various subunits such that the EF-Ts and EF-Tu subunits are co-expressed prior to expression of the β subunit and preferably such that the expression levels of the EF-Ts and EF-Tu subunits can be regulated so as to be uniform.

A variety of inducible promoter systems may be employed in accordance with the invention as will be readily appreciated by those skilled in the art. As exemplified herein two different inducible operons may be employed in order to time the expression of EF-Ts and EF-Tu and β subunit. For example, the use of IPTG induction of the T7 promoter/lac operator to drive expression of the EF-Ts and EF-Tu subunits allows for adjustable and uniform levels of these subunits. Similarly, as exemplified herein expression of the β subunit can be tightly regulated via the araBAD promoter. Other inducible, promoter systems are known to those skilled in the art.

Typically in accordance with the present invention the vectors contemplated are plasmids, although any vectors may be employed provided they are suitable to support expression of the required subunits, and in particular differential temporal expression of the EF-Ts/EF-Tu subunits and the β subunit, and the uniform control of expression levels for the EF-Ts and EF-Tu subunits. Host cells for use in accordance with the invention may be any suitable host cells, typically prokaryotic cells, capable of supporting the differential temporal expression of multiple vectors. In one embodiment the host is E. coil, for example lacZY mutant strains which allow for the regulation and adjustment of protein expression levels throughout all cells in the culture. One exemplary suitable strain is E. coli Tuner® (DE3). In an alternative embodiment, cells containing a chromosomal insertion of the T7 RNA polymerase gene under control of a promoter such as the proU promoter may be used, for example E. coli BL21-S1 cells. In this case, addition of NaCl to the growth medium will induce expression of T7 RNA polymerase, and consequently, genes cloned behind a T7 promoter, enabling expression to be used from this promoter in the absence of IPTG.

The determination and implementation of appropriate conditions to facilitate expression of the required replicase subunits using vectors and host cells as disclosed herein is well within the capabilities and knowledge of those skilled in the art. By way of example, those skilled in the art will appreciate that various parameters of cell culture conditions such as the concentration of any promoter inducers and/or repressors and the temperature at which cells are cultured, may be manipulated in order to achieve the desired outcome.

The present invention also provides kits for carrying out the methods of the invention. In one embodiment, a kit of the present invention comprises (a) a first expression vector comprising polynucleotides encoding the EF-Tu and EF-Ts subunits operably linked to the same or different copies of a first inducible promoter, (b) a second expression vector comprising a polynucleotide encoding the β subunit operably linked to a second inducible promoter, and (c) instructions for expressing the subunits such that expression of the EF-Ts and EF-Tu subunits occurs prior to expression of the β subunit. A kit of the invention may additionally include other components for performing methods of the invention including, for example, DNA sample preparation reagents, reaction buffers, lysis buffers, storage buffers, salts, enzymes, host cells and/or purification columns and appropriate buffers and solutions. The kit may further include reagents for purification of the enzyme. Kits of the invention may further include the necessary reagents for carrying out assays employing the enzyme, such as reagents for replicating and/or amplifying RNA.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to specific examples, which should not be construed as in any way limiting the scope of the invention.

### Examples

PCR primers used in the Examples following are listed in Table 1.

TABLE 1

| | SEQ | |
|---|---|---|
| Primer | ID No. | Sequence^{a} |
| tsfF | 1 | 5′ GAGGATTTTACCATGGCTGAAATTACCGCAT |
| | | C 3′ |
| tsfR | 2 | 5′ CAGGCGGCTCCTTGGATCCAATTAAGACTGC |
| | | T 3′ |
| tufAF | 3 | 5′ GTAAGGAATATACATATGTCTAAAGAAAAAT |
| | | TT 3′ |
| tufAR | 4 | 5′ TCAAAACTAATTAGAGCTCAATTAGCCCAGA |
| | | AC 3′ |
| pBADHisBF | 5 | 5′ GAGACTGCCATTCATGAGTAAGACAGCATCT |
| | | TCG 3′ |
| pBADHisBR | 6 | 5′ ATGCTTAGTGGTGGTGGTAAGCTTACGCCTC |
| | | GTGTA 3′ |

^{a}Engineered restrictions sites are underlined.

### Example 1

### Construction of Recombinant pACYCDuet-TsTu Plasmid

The E. coli genes tsf and tufA encoding the elongation factors EF-Ts and EF-Tu, respectively, were amplified by PCR from E. coli DH5α genomic DNA. tsf was amplified using the specific primers tsfF (SEQ ID No. 1) and tsfR (SEQ ID No. 2) (see Table 1). These primers were designed to include the NcoI and BamHI restriction sites, respectively, which allowed directional in-frame ligation of the amplified tsf PCR fragment into pACYCDuet vector (Novagen). This resulted in recombinant plasmid pACYCDuet-Ts.

The tufA gene was amplified using the primers tufAF (SEQ ID No. 3) and tufAR (SEQ ID No. 4) (see Table 1). These primers incorporate NdeI and SacI restriction sites, respectively, into the amplification product which allowed directional in-frame ligation into vector pET22b (Novagen). This resulted in recombinant plasmid pET22b-Tu. The tufA fragment was excised from pET22b-Tu with NdeI and XhoI and ligated into pACYCDuet-Ts, resulting in recombinant plasmid pACYCDuet-TsTu. Both strands of the recombinant plasmid were sequenced in order to confirm that there were no PCR-derived base changes in the DNA except those introduced in the engineered restriction sites of the PCR primers (Val→Met at the beginning of the EF-Tu peptide).

### Example 2

### Construction of Recombinant pBAD-β Subunit Plasmid

The Qβ replicase β-subunit gene was kindly provided by Dr. Y. Inokuchi, Teikyo University, Japan. The β-subunit gene was re-engineered and ligated into the expression vector pJLA602 by Dr. R. Anitori to produce recombinant plasmid pJLA602-Qβ. The β subunit gene was PCR amplified from pJLA602-Qβ using the primers pBADHisBF (SEQ ID No. 5) and pBADHisBR (SEQ ID No. 6) (see Table 1). These primers were designed to incorporate BspHI and HindIII restriction sites, respectively into the amplification product. Further, plasmid pJLA602-Qβ carries a DNA fragment encoding a C-terminal Hiss-tagged form of the Qβ replicase β subunit, however the His₆-tag sequence was eliminated in the PCR amplification. The PCR amplified β subunit fragment without C-terminal Hiss-tag was ligated into the NcoI and HindIII restriction sites of plasmid pBAD/His B (Invitrogen). This resulted in recombinant plasmid pBAD-βsubunit. Both strands of the recombinant plasmid encoding the β subunit were sequenced in order to confirm that there were no FOR-derived base changes in the DNA.

### Example 3

### Production of Qβ Replicase Heterotrimer

E. coli Tuner® (DE3) cells (Novagen) were used for co-expression experiments of EF-Ts, EF-Tu and β-subunit. E. coil Tuner® competent cells were transformed with recombinant plasmid pACYCDuet-TsTu. Cells carrying the recombinant plasmid pACYCDuet-TsTu were then made competent by the method of Chung (1989) and transformed with plasmid pBAD-βsubunit.

For the production of recombinant Qβ replicase, 500 ml Luria Bertani (LB) medium without antibiotics, was inoculated with 1 ml of an overnight culture (supplemented with 50 μg/ml ampicillin and 34 μg/ml chloramphenicol) of E. coli Tuner® (DE3) harbouring the recombinant pACYCDuet-TsTu and pBAD-βsubunit plasmids. The culture was incubated at 37° C., with shaking (250 rpm), until the A₆₀₀ was between 1 and 2. EF-Ts and EF-Tu protein synthesis was then induced by the addition of 1 mM IPTG. Subsequently, β subunit protein synthesis was induced 30 min after the IPTG induction by addition of arabinose at a final concentration of 0.2%, while the temperature was reduced to 30° C. Cells were harvested after 2 h incubation by centrifugation for 15 min at 10,000×g and 4° C. Cells were stored at −20° C.

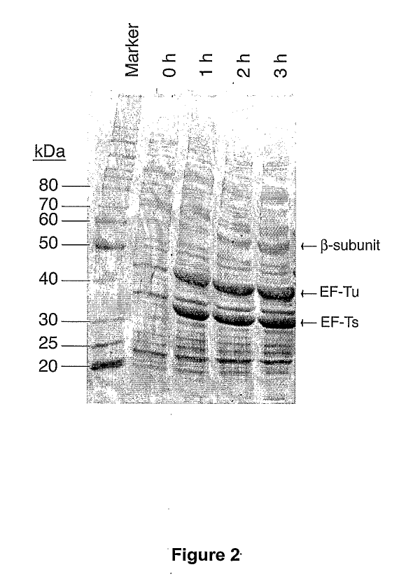

Culturing of cells to maintain both plasmids pACYCDuet-TsTu and pBAD-βsubunit typically requires a growth medium containing chloramphenicol and ampicillin. However, this can result in plasmid amplification, and indeed preliminary attempts to over-express the β subunit under these conditions resulted in mostly insoluble protein, being produced (data not shown). In order to decrease plasmid amplification, co-expression was carried out in growth medium without antibiotics. As shown in FIG. 1, after a short IPTG induction period of 30 min, high levels of EF-Ts and EF-Tu were expressed in the E. coli Tuner (DE3) cells. The E. coli Tuner strain allows the induction with IPTG in a concentration-dependent manner throughout all cells in the population. This pre-expression of EF-Ts and EF-Tu appears to be a pre-requisite for the proper folding of the β-subunit. The β subunit expression with 0.2% arabinose at 30° C. was initiated 30 min after EF-Ts and EF-Tu induction. High levels of β subunit were detected after 2 h induction. The pBAD promoter allows expression of the β subunit under tight regulation. Thus, no detectable levels of recombinant β-subunit were observed prior to arabinose induction (FIG. 2).

### Example 4

### Purification of Qβ Replicase

All buffers and reagents used for purification were RNase and DNase free. Cells cultured as described in Example 3 were resuspended in buffer A (100 mM phosphate, pH 8.2, 500 mM NaCl, 20% glycerol, 5 mM MgCl₂, 5 mM 2-mercaptoethanol and 1 mM EDTA) and supplemented with 50 U DNase I (Invitrogen) and 2.0 mg lysozyme (Sigma). The cells were ruptured by sonication. The debris was removed by centrifugation for 30 min at 20,000×g and 4° C. The supernatant obtained was buffer exchanged using a PD-10 desalting column (GE Healthcare) equilibrated with buffer B (50 mM Tris-HCl, pH 8.0, 100 mM NaCl, 5 mM MgCl₂, 1 mM EDTA, and 5 mM 2-mercaptoethanol).

The extract was loaded onto a 5 ml HiTrap Q anion exchanger column (GE Healthcare) previously equilibrated with buffer B and washed extensively with buffer B containing 150 mM NaCl. Bound proteins were eluted with 250 mM NaCl. The eluted proteins were diluted with buffer to a final concentration of 100 mM NaCl. Then, the sample was applied to a 5 ml HiTrap SP cation exchanger column (GE Healthcare) previously equilibrated with buffer B containing 100 mM NaCl. The column was extensively washed with the same buffer and the Qβ replicase heterotrimer was eluted with 250 mM NaCl. Fractions containing Qβ replicase were identified by SDS-PAGE and staining with Coomassie brilliant blue, pooled and concentrated using an Amicon Ultra-15 centrifugal filter (50 kDa cut-off, Millipore). Samples were stored in 50 mM Tris-HCl, pH 8.0, 100 mM NaCl, 1 mM DTT, 0.1 mM EDTA, 0.1% Triton X-100 and 50% glycerol, at −20° C.

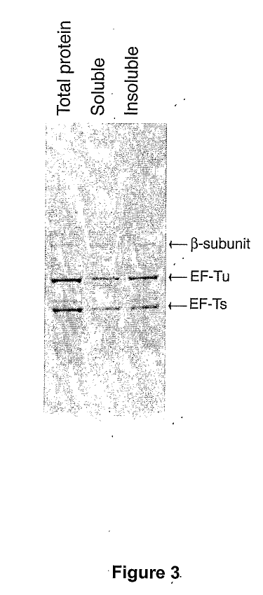

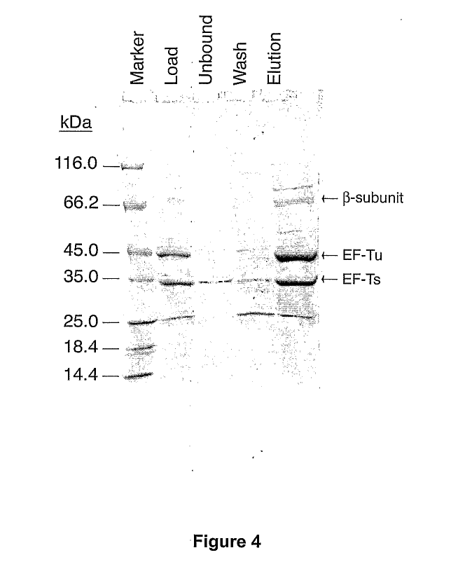

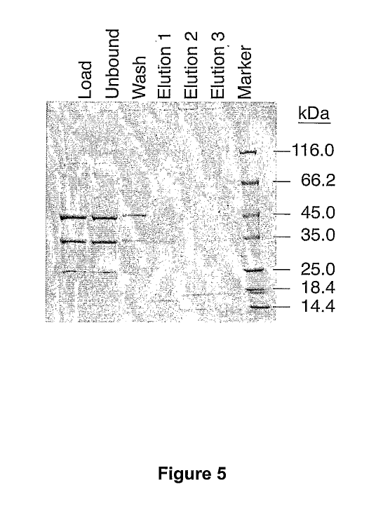

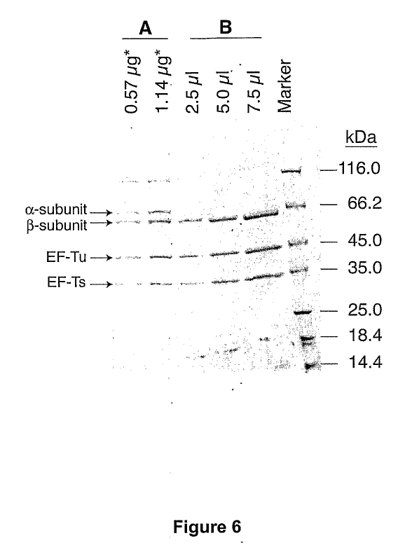

Recombinant Qβ replicase heterotrimer complex was purified as described above to electrophoretic homogeneity. Sonication treatment of the cells harvested after co-expression indicated that at least 50% of the β subunit, EF-Ts and EF-Tu are present in the soluble fraction (FIG. 3). All three subunits were eluted from the HiTrap Q anion exchanger column with 250 mM NaCl (FIG. 4). The eluted fraction contained an excess of the over-expressed EF-Ts and EF-Tu. The NaCl concentration of the eluted fraction was diluted to 100 mM and then loaded into a HiTrap SP cation exchanger column. The excess of EF-Ts and EF-Tu (not part of the Qβ replicase heterotrimer complex) was found mainly in the unbound and wash fractions (FIG. 5). The Qβ replicase heterotrimer complex, containing EF-Ts, EF-Tu and β subunit in equal proportions, was eluted with 250 mM NaCl (FIG. 4). (Elutions 1, 2 and 3 as represented in FIG. 5 are three consecutive elutions of approximately 15 ml each using buffer B+250 mM NaCl.) Finally, small molecular weight contaminants present in the eluted fraction were removed by concentration on an Amicon Ultra-15 centrifugal filter (50 kDa cut-off, Millipore). The quality of the final purified Qβ replicase heterotrimer enzyme preparation (B) is demonstrated alongside a commercial Qβ replicase heterotetramer (Epicentre Biotechnologies) control (A) in FIG. 6.

### Example 5

### Modified Production Protocol for Qβ Replicase Heterotrimer and Purification Thereof

Modifications were made to the production protocol described in Example 3, specifically utilizing a reduced concentration of IPTG and a reduced expression temperature.

E. coli cells and plasmids and cellular transformations were as described in Example 3. Subsequently, 250 ml Luria Bertani (LB) medium without antibiotics, was inoculated with 0.5 ml of an overnight culture (supplemented with 50 μg/ml ampicillin and 34 μg/ml chloramphenicol) of E. coil Tuner® (DE3) harbouring the recombinant pACYCDuet-TsTu and pBAD-βsubunit plasmids. The culture was incubated at 37° C., with shaking (250 rpm), until the A₆₀₀ was between 0.6 and 0.8. The incubation temperature was reduced to 20° C. and EF-Ts and EF-Tu protein synthesis was then induced by the addition of 0.2 mM IPTG. Subsequently, β subunit protein synthesis was induced 1 h after the IPTG induction by addition of arabinose at a final concentration of 0.2%. Cells were harvested after 2 h incubation by centrifugation for 15 min at 10,000×g and 4° C. Cells were stored at −20° C.

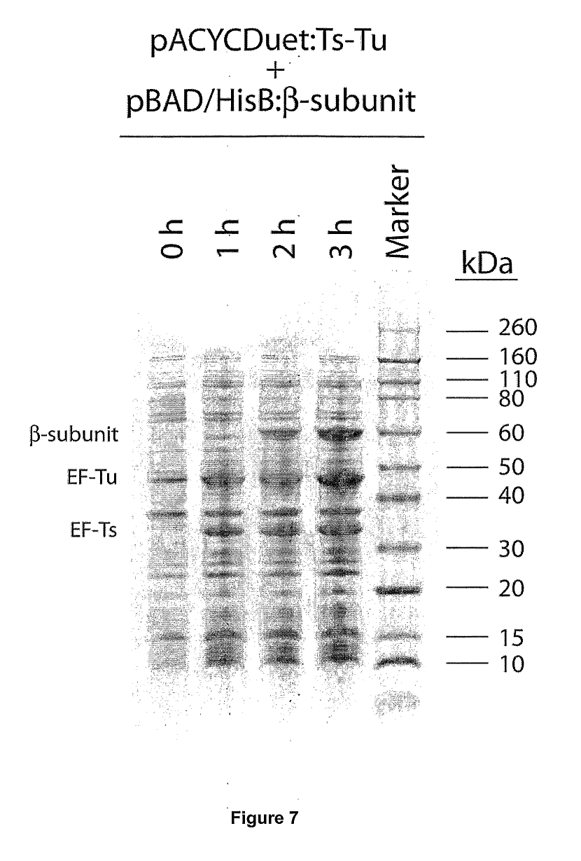

As shown in FIG. 7, reducing the concentration of IPTG from 1 mM (Example 3) to 0.2 mM and the lowering the overall expression temperature from 30° C. (Example 3) to 20° C. had a direct effect on the expression levels of EF-Ts and EF-Tu. Under these modified conditions there was a decrease in the total amount of. EF-Ts and EF-Tu expressed, while the levels of β-subunit increased. Furthermore, all three subunits (EF-Ts, EF-Tu and [β-subunit) were expressed in the E. coli Tuner (DE3) cells at comparable levels. Achieving comparable expression levels of all three subunits is advantageous for the production of Qβ replicase since the three subunits are present in the heterotrimer in 1:1:1 stoichiometry (ratio). Thus, by having comparable expression levels for all three subunits, the yields of the final heterotrimer is improved.

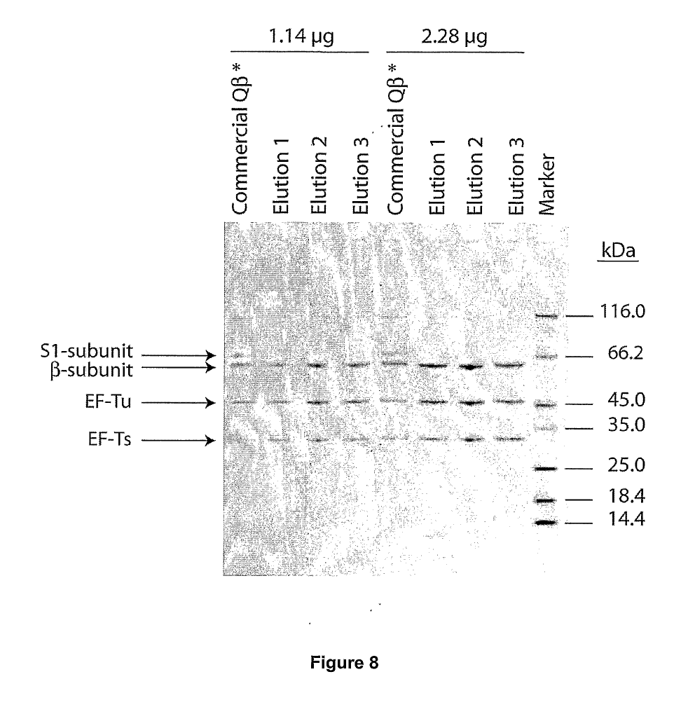

The Qβ replicase heterotrimer produced using the above protocol was purified using a HiTrap S cation-exchange column (GE Healthcare) as described in Example 4. SDS-PAGE analysis of the purified heterotrimer complex is shown in FIG. 8. The Qβ replicase complex was eluted from the HiTrap S cation-exchange column with 250 mM NaCl (Elution). The three fractions obtained at 250 mM NaCl were concentrated and buffer-exchanged using an Amicon Ultra-15 centrifugal filter (Millipore) as described in Example 4. A commercial Qβ replicase heterotetramer (Epicentre Biotechnologies) was included for comparative purposes.

### Example 6

### Activity of the Purified Qβ Replicase Heterotrimer

The purified Qβ replicase heterotrimer complex (as described in Example 4) was assayed for replicase activity using midivariant (MDV)-poly(+) RNA as a template. The reaction mixture (50 μl) contained 0.8 mM each of ATP, CTP, GTP and UTP, 600 ng MDV-1 RNA, 125 mM Tris-HCl (pH 8.0), 20 mM MgCl₂, 25 mM 2-mercaptoethanol, 5 mM phosphoenol pyruvate, 10 μg/ml pyruvate kinase, 74 U/ml DNase I, 10 μg/ml rifampicin, 1 U/μl RNasin and 2.5-5.0 μl of purified enzyme sample. The reaction was carried out for 30 min at 35° C. The reaction was stopped with 20 mM EDTA (pH 8.0). The ethanol precipitated RNA was dissolved in 7 μl water and analysed on denaturing formaldehyde/MOPS (4-morpholinepropanesulfonic acid) agarose-gels. RNA was stained with SYBR Green II (Invitrogen). Commercial Qβ replicase heterotetramer was used as a positive control in the assays.

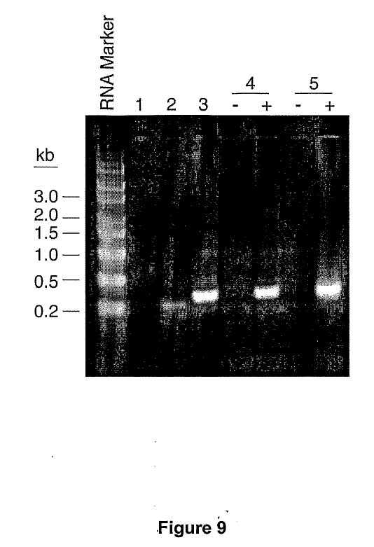

In the absence of template RNA no product formation was observed with either the commercial or the purified replicase indicating the lack of contaminant RNA in the purified replicase preparation (FIG. 9). When midivariant (MDV)-poly(+) RNA template was present in the assay reaction, both the commercial and purified replicases produced large amounts of amplified RNA product (see FIG. 9).

1. A method for producing a replicase comprising EF-Ts, EF-Tu and β polypeptide subunits, the method comprising:
(a) expressing in a suitable host cell the EF-Ts and EF-Tu subunits;
followed by
(b) expressing the β subunit,
wherein the EF-Ts and EF-Tu subunits are operably linked to a first promoter and the β subunit is operably linked to a second promoter, and wherein the first and second promoters are differentially induced.

2. The method of claim 1 wherein a dimeric EF-TsTu complex is formed from step (a) prior to expression of the β subunit in step (b).

3. The method of claim 1 wherein the EF-Ts and EF-Tu subunits are encoded by polynucleotides located in a first vector, and the polynucleotide encoding the β subunit is located in a second vector.

4. The method of claim 3 wherein the first vector comprises at least two copies of the first promoter and the polynucleotides encoding the EF-Ts and EF-Tu subunits are operably linked to different copies of the first promoter.

5. The method of claim 1 wherein the first promoter is IPTG-inducible and the second promoter is arabinose-inducible or vice versa.

6. The method of claim 1 wherein the replicase is the RNA-dependent RNA polymerase of a coliphage of the alloleviridae.

7. The method of claim 6 wherein the coliphage is phage Qβ or phage SP.

8. The method of claim 7 wherein the coliphage is phage Qβ.

9. The method of claim 1 wherein the replicase is the RNA-dependent RNA polymerase of a coliphage of the leviviridae.

10. The method of claim 9 wherein the coliphage is phage MS2 or phage GA.

11. The method of claim 1 wherein the resulting replicase is a heterotetrameric enzyme further comprising an α subunit.

12. The method of claim 1 comprising the additional step of purifying the replicase from the host cell.

13. A method for the production of replicase heterotrimer comprising EF-Ts, EF-Tu and β polypeptide subunits, the method comprising:
(a) providing a first expression vector comprising polynucleotides encoding the EF-Ts and EF-Tu subunits operably linked to the same or different copies of a first inducible promoter;
(b) providing a second expression vector comprising a polynucleotide encoding the β subunit operably linked to a second inducible promoter, wherein the first and second promoters are differentially inducible;
(c) transforming a suitable host cell with both the first and second expression vectors;
(d) culturing host cells under conditions suitable to allow expression of the EF-Ts and EF-Tu subunits from the first expression vector; and
(e) subsequently culturing host cells under conditions suitable to allow expression of the β subunit from the second expression vector.

14. The method of claim 13 wherein a dimeric EF-TsTu complex is formed from step (a) prior to expression of the β subunit in step (b).

15. The method of claim 13 further comprising the purification of the replicase from the host cells.

16. The method of claim 15 wherein the purification comprises the steps of:
(a) lysing the host cells and collecting the supernatant containing the replicase;
(b) applying the extract supernatant to an anion exchange column;
(c) eluting one or more fractions from the column containing the replicase;
(d) applying the fractions from (c) to a cation exchange column; and
(e) eluting purified replicase from the column.

17. A recombinant replicase produced in accordance with the method of claim 1.

18. A dual vector expression system for use in the production of a replicase heterotrimer comprising EF-Ts, EF-Tu and β polypeptide subunits, the system comprising a first expression vector comprising polynucleotides encoding the EF-Tu and EF-Ts subunits operably linked to the same or different copies of a first inducible promoter and a second expression vector comprising a polynucleotide encoding the β subunit operably linked to a second inducible promoter, wherein the first and second promoters are differentially inducible, and wherein in use, the EF-Ts and EF-Tu subunits are expressed prior to the β subunit.
